(19) United States
(12) Patent Application Publication
(10) Pub. No.: US 2026/0166276 A1
(43) Pub. Date: Jun. 18, 2026

(54) CUSTOMIZABLE SELF-PACKED MEDICAL DEVICE KIT

(71) Applicant: Becton, Dickinson and Company, Franklin Lakes, NJ (US)

(72) Inventors: Shishir Prasad, Ramsey, NJ (US); Eric Dustin Agdeppa, Cary, NC (US); Lance Hussey, Simi Valley, CA (US); Cary Chow, Santa Monica, CA (US); Ravi Sawhney, Malibu, CA (US); Bridget Bergman, Lake Forest, CA (US); Delaney Santos, Camarillo, CA (US); Elizabeth Huang, Fountain Valley, CA (US); Dean Loock, North Hollywood, CA (US)

(73) Assignee: Becton, Dickinson and Company, Franklin Lakes, NJ (US)

(21) Appl. No.: 19/129,576

(22) PCT Filed: Dec. 18, 2023
(86) PCT No.: PCT/US2023/084591

§ 371 Date: May 13, 2025

Related U.S. Application Data
(60) Provisional application No. 63/433,658, filed on Dec. 19, 2022.

(51) Int. Cl. A61M 25/01

(52) U.S. Cl. CPC A61M 25/0113

(57) ABSTRACT

Provided herein is a medical device kit and method of providing such a medical device kit. A kit housing is initially provided in an empty state, with the kit housing including a plurality of receptacles therein configured to store medical supplies. The kit housing is then packed with medical supplies, with the medical supplies inserted into the receptacles of the kit housing. A sterile prep sheet is then prepared and packaged in the kit housing, with the sterile prep sheet including thereon queuing areas and instructions for use for some or all of the medical supplies. The kit housing is then actuated into a storage configuration.

### CROSS-REFERENCE TO RELATED APPLICATION

The present application claims priority to U.S. Provisional Patent Application No. 63/433,658 entitled “Customizable Self-Packed Medical Device Kit” filed Dec. 19, 2022, the entire disclosure of which is hereby incorporated by reference in its entirety.

### BACKGROUND OF THE INVENTION

### Field of the Invention

The present disclosure relates generally to kits for use in placing, maintaining, altering, and/or removing medical devices in, on, and/or from the body of a patient.

### Description of Related Art

Medical device kits (or “convenience kits”) are used by medical care providers and clinicians for various medical treatments or surgical procedures, with procedures such as intravascular (IV) catheter insertion, IV catheter maintenance, and anesthesia being examples. A medical device kit packages together all the medical devices and products that are necessary for the particular treatment or procedure that is to be performed. As one example, catheter kits are available in the market for catheter dressing, catheter flushing, catheter insertion, and catheter removal. Catheter kits, depending on the exact use or application thereof, may include at least some of sterile gloves, alcohol wipes, syringes, gauze, sutures, tape, dressings, disinfecting caps, swab sticks, tourniquets, securement devices, hemostatic agents, and/or anticoagulants, as examples.

Existing medical device kits, including catheter kits as described above, typically have no provision for customization of the medical supplies included therein. That is, while there may be a multitude of medical device kits available from kit manufacturers for a given procedure, each of the medical device kits comes pre-packaged with a standard collection of devices and products therein. It is recognized, however, that certain clinicians or other healthcare workers—when performing a medical treatment or surgical procedure using an appropriate medical device kit—may prefer or desire a device or product not provided in the kit. For example, if a second set of gloves is required or an alternate type/brand of tourniquet is preferred by the clinician, then the clinician must either open a new kit or need to separately order the preferred brand of product. In either case, a device or product in a medical device kit may be left unused. As the established medical practice is for clinicians to discard all unused devices/products in a medical device kit opened at the patient bedside, it can be seen that the use of standard, pre-packaged medical device kits may lead to a large amount of unnecessary waste being generated and disposed of. This may even be the situation in the case where a healthcare facility decides to standardize the medical devices required for a procedure or carries multiple kits for the procedure.

Accordingly, a need exists in the art for medical device kits that may be customizable to the particular desires and needs of a clinician or healthcare facility, whereby the disposal of unwanted or unnecessary devices or components from a pre-packaged medical device kit that go unused can be eliminated. It would also be desirable for such kits to include features therein that provide assistance regarding the organization and use of the devices or components included in the customized medical device kit, so as to improve efficiency of the procedure being performed.

### SUMMARY OF THE INVENTION

Provided herein is a medical device kit for packaging medical supplies for use in a medical procedure. The medical device kit includes a housing sheet having an inner surface and an outer surface, with the housing sheet configurable between a usage configuration and a storage configuration. The medical device kit also includes a plurality of pockets attached to the inner surface of the housing sheet and configured to retain medical supplies therein, a sterile prep sheet positioned in a pocket of the plurality of pockets and including queuing areas for arranging the medical supplies and instructions for use for some or all of the medical supplies, and a fastening device configured to secure the housing sheet in the storage configuration.

In some embodiments, the sterile prep sheet is pre-labeled with the instructions for use, to enable proper arrangement of medical supplies in the medical device kit on the queuing areas.

In some embodiments, the instructions for use are ordered sequentially, according to an order in which the medical supplies are to be used in the medical procedure.

In some embodiments, the medical device kit is a customizable, self-packed medical device kit, with the medical supplies comprising a customized collection of medical supplies packed by a user, as determined by the medical procedure.

Also provided herein is a medical device kit for packaging medical supplies for use in a medical procedure. The medical device kit includes a box-type housing defining an interior volume, with the box-type and having a lid movable between an open position and a closed position, and with the box-type housing being in the storage configuration when the lid is in the closed position. The medical device kit also includes one or more partitions positioned within the interior volume that define a plurality compartments in the box-type housing configured to receive medical supplies therein and a sterile prep sheet attached to an inner surface of the lid and including queuing areas for arranging the medical supplies and instructions for use for some or all of the medical supplies.

In some embodiments, the sterile prep sheet is attached to the inner surface of the lid in a folded configuration, so that an inner surface of the sterile prep sheet is not exposed.

In some embodiments, the sterile prep sheet is pre-labeled with the instructions for use, to enable proper arrangement of medical supplies in the medical device kit on the queuing areas.

In some embodiments, the instructions for use are ordered sequentially, according to an order in which the medical supplies are to be used in the medical procedure.

Also provided herein is a method of providing a medical device kit having packaged therein medical supplies for use in a medical procedure. The method includes storing, in a memory, a plurality of kit housing types, wherein a kit housing type indicates a set of medical supplies that can be stored by a kit housing of the kit housing type. The method also includes receiving an identifier for a treatment facility, determining medical supplies stocked at the treatment facility, identifying a kit housing type of the plurality of kit housing types that can store at least a portion of the medical supplies stocked at the treatment facility; and providing a kit housing of the kit housing type to the treatment facility.

In some embodiments, the method also includes providing a kit housing in an empty state, the kit housing comprising a plurality of receptacles provided therein configured to store medical supplies therein, receiving the medical supplies into respective ones of the plurality of receptacles, receiving a sterile prep sheet in the kit housing that includes queuing areas and instructions for use for some or all of the medical supplies received by the kit housing, and actuating the kit housing into a storage configuration.

In some embodiments, providing the kit housing includes providing a housing sheet configurable between a flat position and a rolled position, with the housing sheet being in the storage configuration when in the rolled position, and wherein the plurality of receptacles comprise a plurality of pockets provided on the housing sheet.

In some embodiments, receiving the medical supplies and receiving the sterile prep sheet includes inserting the medical supplies and the sterile prep sheet into the plurality of pockets.

In some embodiments, actuating the kit housing into the storage configuration includes securing the housing sheet in the rolled position via securing of a fastening device on the housing sheet.

In some embodiments, providing the kit housing includes providing a box-type housing having a lid movable between an open position and a closed position, with the box-type housing being in the storage configuration when the lid is in the closed position, and wherein the plurality of receptacles is a plurality compartments defined in the box-type housing.

In some embodiments, receiving the medical supplies includes depositing the medical supplies into the plurality of compartments, and wherein receiving the sterile prep sheet includes attaching the sterile prep sheet to an inner surface of the lid, the sterile prep sheet attached so as to be in a folded configuration, so that an inner surface of the sterile prep sheet is not exposed.

In some embodiments, providing the kit housing includes providing the box-type housing in a flat, unstructured initial state, and reconfiguring the box into a three-dimensional shape defining the plurality compartments.

In some embodiments, receiving the medical supplies includes selecting a customized collection of medical supplies to pack in the kit housing, as determined by the medical procedure.

In some embodiments, the queuing areas are demarcated areas printed directly on the sterile prep sheet.

In some embodiments, preparing the sterile prep sheet includes pre-labeling the sterile prep sheet with the instructions for use, with the instructions for use included according to a sequential order in which the medical supplies are to be used in the medical procedure.

In some embodiments, the method further includes attaching an identification label to the kit housing, the identification label indicating the medical supplies included in the medical device kit.

In some embodiments, receiving the medical supplies includes self-packing the kit housing with medical supplies at a healthcare facility at which the medical procedure is to be performed.

### BRIEF DESCRIPTION OF THE DRAWINGS

FIG. 1 is a perspective view of a catheter inserted into a body of a patient, exhibiting one example environment with which embodiments of the disclosure can be practiced;

FIG. 2 is a perspective view of a medical device kit, prior to packing of the kit with medical supplies, according to an embodiment described herein;

FIG. 3 is a perspective view of the medical device kit of FIG. 2, with medical supplies packed therein;

FIG. 4 is a perspective view of the medical device kit of FIG. 3, packed and in a storage configuration;

FIG. 5 is a perspective view of the medical device kit of FIG. 3, with a prep sheet unpacked therefrom;

FIG. 6 illustrates disposal of the medical device kit of FIG. 3 after use;

FIG. 7 is a perspective view of a medical device kit, prior to packing of the kit with medical supplies, according to another embodiment described herein;

FIG. 8 is a perspective view of the medical device kit of FIG. 7, with medical supplies packed therein;

FIG. 9 is a perspective view of the medical device kit of FIG. 8, packed and in a storage configuration;

FIG. 10 is a perspective view of the medical device kit of FIG. 8, with a prep sheet of the kit in an unfolded position;

FIG. 11 illustrates disposal of the medical device kit of FIG. 8 after use; and FIG. 12 is a flowchart illustrating a preparation and usage cycle of a self-packed, customizable medical device kit, according to an embodiment described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

As used herein, the term “computing device” may refer to one or more electronic devices that are configured to directly or indirectly communicate with or over one or more networks. A computing device may be a mobile or portable computing device, a desktop computer, a server, and/or the like that is specifically configured to provide one or more of the features and/or functions described herein. Furthermore, the term “computer” may refer to a specifically configured computing device that includes the necessary components to receive, process, and output data according to aspects described herein, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, firmware, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Provided herein are medical device kits for use in placing, maintaining, altering, and/or removing medical devices in, on, and/or from the body of a patient. Such medical device kits can include a plurality of medical devices and supplies for use in the placement/maintenance procedure. In accordance with present embodiments, the medical device kits are customizable kits that can be self-packed according to the desired specifications of a healthcare provider, with the medical device kits including various organizational and usage features to assist the clinician or user performing the particular procedure. These various features are outlined in further detail below.

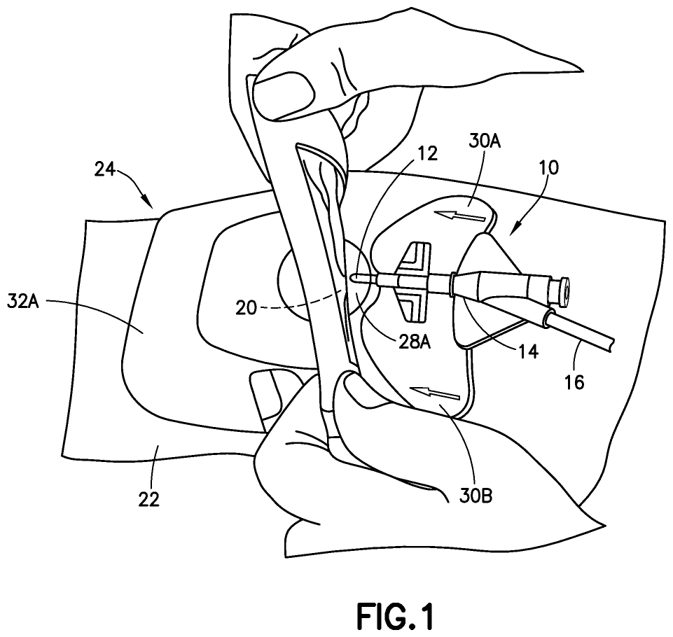

Reference is first made to FIG. 1, which depicts a catheter 10 including a catheter tube 12 inserted transcutaneously through a skin 22 of a patient 24 via an insertion site 20. Though the catheter 10 shown here is a peripheral intravenous catheter (PIVC), various other catheters and medical devices can benefit from the principles and features described below. Examples of other catheters and elongate tubular devices include dialysis catheters, Foley and urinary catheters, feeding tubes, balloon catheters, PIVs, etc. Vascular and other types of access ports are further examples of medical devices that may be employed. Also, though shown here inserted into the arm of the patient 24, the catheter 10 or other medical device can be disposed in other areas of the body of the patient, not only transcutaneously, but topically or subcutaneously as well. In another embodiment, the medical device is not connected to or in contact with the patient's body. Thus, these and other modifications are therefore contemplated.

FIG. 1 further shows that the catheter 10 includes an adapter 14 and one or more fluid conduits 16 that operably connect, via the adapter 14, to a corresponding number of lumens defined by the catheter tube 12. An antimicrobial/hemostatic patch 28A, is disposed about the catheter tube 12 at the insertion site 20, and a catheter securement device 30A is removably attached to the patient skin 22 so as to removably attach to and secure the adapter 14 of the catheter. An adhesive dressing 32A is adhesively and removably positioned so as to cover the external portion of the catheter 10, the patch 28A, and the securement device 30A and isolate the insertion site 20 and protect it from contamination. The patch 28A, the securement device 30A, and the adhesive dressing 32A are collectively referred to herein as a “dressing assembly” or “dressing,” though it is appreciated that more or fewer components can be included in the assembly.

As shown in FIG. 1, it is periodically necessary to change the current/existing dressing assembly of the indwelling catheter 10 and replace it with a new dressing assembly. As such, FIG. 1 shows the patch 28A, the securement device 30A, and the adhesive dressing 32A as old components needing to be changed, with FIG. 1 further showing the old adhesive dressing 32A being removed from the patient skin 22 by a clinician. Additionally, it is recognized that additional procedures related to use of an indwelling catheter 10 may need be performed, including flushing of the catheter 10, removal of the catheter 10, and/or insertion of a catheter 10 (i.e., an initial insertion of the catheter 10 or removal of an existing catheter and replacement with a new catheter 10).

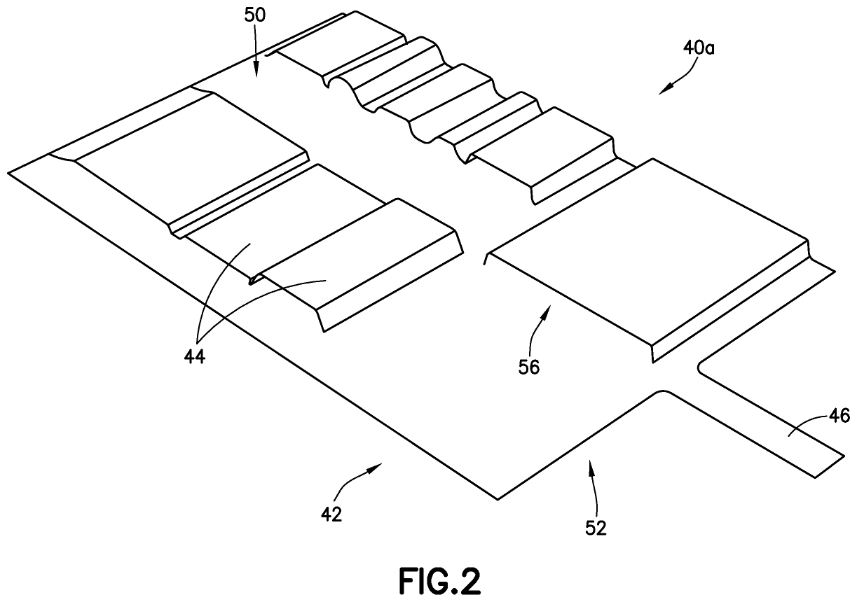

FIGS. 2-11 show a medical device kit (“kit”) 40 according to various embodiments, which can be used in a procedure to replace the old dressing assembly with a new dressing assembly for an indwelling catheter, such as the catheter 10 shown in FIG. 1. In other embodiments, and as indicated above, the kit 40 can be used in a procedure to flush an indwelling catheter, remove an indwelling catheter, or insert a new indwelling catheter. The kit 40 of FIGS. 2-11 is thus described in the present embodiment as a catheter kit or catheter maintenance kit and includes various components needed to remove or insert a catheter or perform catheter maintenance (dressing change procedure, catheter flushing procedure, etc.), though it is appreciated that the kit 40 can be configured for one or more of a variety of procedures involving a medical device (e.g., anesthesia kit). The discussion to follow is therefore illustrative and is not to be considered as limiting.

As described in detail below, in according with aspects of the disclosure, a kit 40 is initially provided as an empty housing or casing that does not include any medical devices or components pre-packaged therein. With the kit 40 provided initially as an empty housing, a healthcare worker may pack any medical devices or components into the kit 40 deemed necessary for a procedure to be performed by a clinician, so as to provide a self-packed, “customized” kit 40. Accordingly the kit 40 may include only those medical devices or components desired for the procedure to be performed by the clinician, eliminating the potential for a device or component to go unused and be subsequently disposed of.

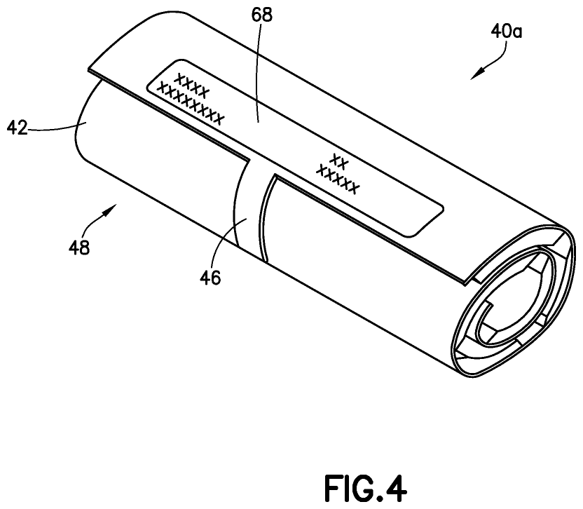

Referring first to FIGS. 2-6, a kit 40a is shown in accordance with one aspect of the disclosure. The kit 40a generally includes a housing 42 and a plurality of medical supplies packaged in the housing. As can be seen in FIG. 2, the kit 40a is provided initially as an empty housing 42, with the housing 42 in the form of a housing sheet 42 that includes a plurality of pockets 44 and a fastening device 46 thereon. The housing sheet 42 may be employed, in one embodiment, to serve as an outer housing that protects and supports the components of the kit 40a when the kit 40a is in a storage configuration 48. In one embodiment, the housing sheet 42 is configured to be rolled up into a cylindrical shape to present the kit 40a in the storage configuration 48 (FIG. 4); however, it is recognized that the housing sheet 42 could instead be foldable in a number of different arrangements to present the kit 40a in the storage configuration 48, according to other embodiments.

In greater detail, the housing sheet 42 is configured as a substantially flat, flexible sheet or body. The housing sheet 42 may thus be formed of a spunbond-meltblown-spunbond (“SMS”) nonwoven fabric or other suitable material/fabric that, in some embodiments, is a waterproof nonwoven fabric. The housing sheet 42 includes an inner surface 50 and an opposite outer surface 52, with the inner and outer surfaces 50, 52 being understood as the surfaces that face inward (inner surface 50) and outward (outer surface 52) when the kit 40a is in a rolled, storage configuration 48 and that face upward (inner surface 50) and downward (outer surface 52) when the kit 40a is in an unrolled, usage configuration 54.

A plurality of pockets 44 are included in the kit 40a for receiving therein the medical devices and supplies that are to be used during the procedure to be performed by the clinician, such as removing the old dressing assembly from the catheter 10. The pockets 44 are positioned on the inner surface 50 of the housing sheet 42. In some embodiments, the pockets 44 are formed of a polymeric material (e.g., polypropylene), with each pocket 44 shaped to form a receptacle therein having an opening 56 on one side. The pockets 44 may be secured to the housing sheet 42 via use of an adhesive or other mechanical fastening means, according to embodiments.

As previously indicated, each of the pockets 44 is sized to receive therein medical devices and supplies (generally indicated as 58) that are to be used during the procedure to be performed by the clinician. According to embodiments, the devices/supplies 58 may include sterile gloves, alcohol wipes, syringes, gauze, sutures, tape, dressings, disinfecting caps, swab sticks, tourniquets, securement devices, hemostatic agents, and/or anticoagulants, as examples. The components could vary in number, type, position, etc., in other embodiments, however. Similarly, the size, shape, number, and configuration of the pockets 44 themselves can vary from what is shown and described herein. In the present embodiment, the kit 40a 40 includes eight (8) pockets 44, though other numbers of pockets 44 can be included.

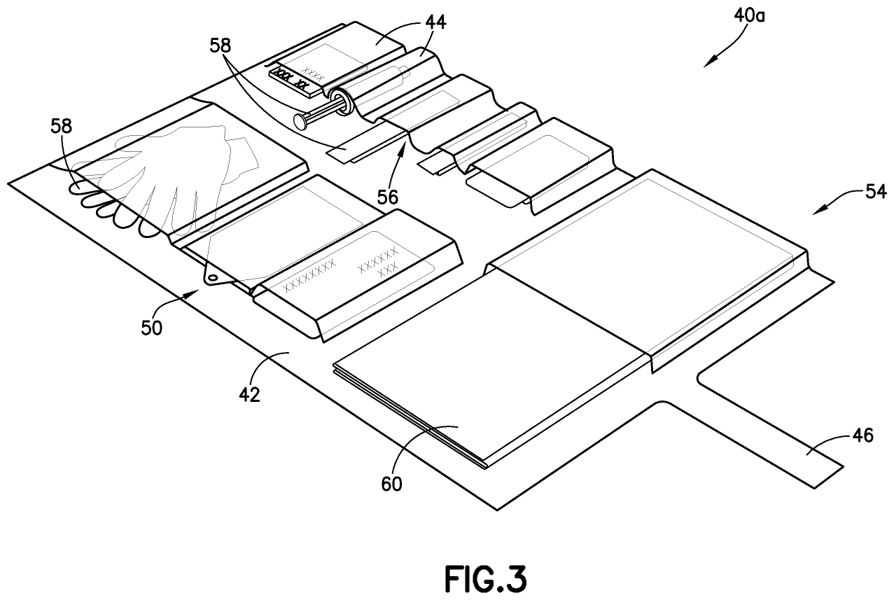

As shown in FIG. 3, one of the pockets 44 is sized and configured to receive therein a prep sheet 60 that, during usage of the kit 40a, may be removed and unfolded to provide a surface/platform or “prep area” on which the medical devices and supplies 58 included in the kit 40a may be placed and arranged by the clinician upon being removed from the pockets 44. In some embodiments, the prep sheet 60 may be subjected to sterilization procedures sufficient to render the prep sheet 60 a sterile field onto which the devices/supplies 58 may be laid (with the devices/supplies 58 also sterilized), while in other embodiments the prep sheet 60 is configured as a clean surface instead of a sterile surface, e.g., the prep sheet 60 is suitable for a procedure where only a clean field is required, as opposed to a sterile field. In other embodiments, the prep sheet 60 can be configured as other field types, including aseptic, in one embodiment.

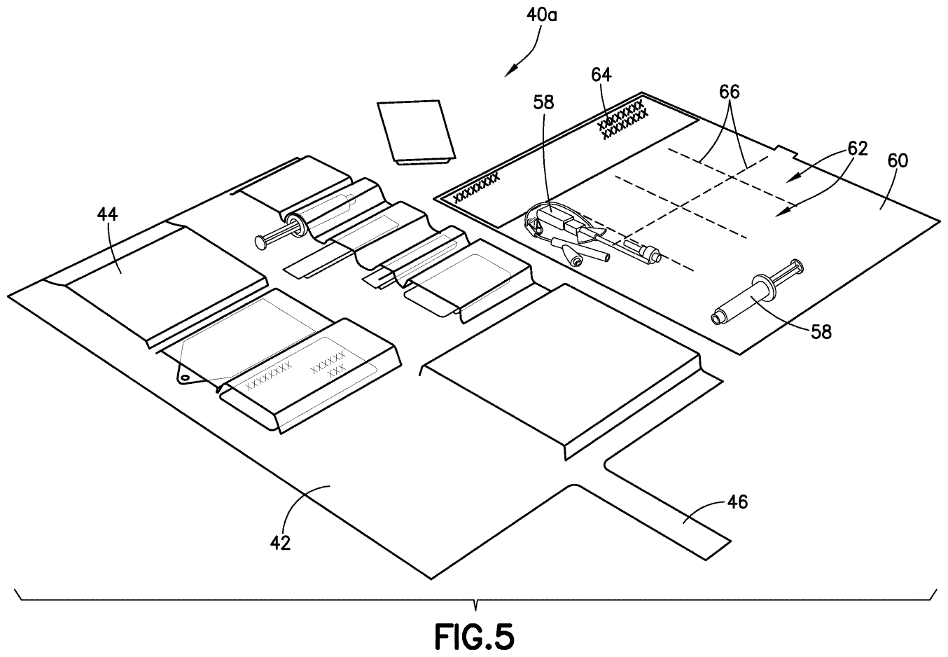

FIG. 5 illustrates the sterile prep sheet 60 in an unfolded state and arranged to receive medical supplies 58 thereon from kit 40a. The prep sheet 60 includes thereon a plurality of queuing areas 62 for arranging the medical supplies 58, as well as instructions for use 64 for some or all of the medical supplies in kit 40a. In some embodiments, the queuing areas 62 are provided as a grid-like arrangement of rectangular areas, with the areas demarcated by lines 66 printed directly on the prep sheet 60. The instructions for use 64 of the various medical supplies 58 may be provided above the queuing areas 62. In some embodiments, the instructions for use 64 may be provided in the form of an adhesive label that may secured on the prep sheet 60. The instructions for use 64 may include detailed instructions thereon for the use of some or all of the medical supplies 58 to be arranged in the queuing areas 62. The instructions for use 64 are provided in a sequential arrangement in accordance with an order in which the medical supplies 58 are to be used in the medical procedure—i.e., a series of sequential steps for performing the procedure, with a medical device or component used at each step.

As shown in FIG. 4, fastening device 46 is provided on kit 40a to secure the kit 40a in the storage configuration 48. According to some embodiments, the fastening device 46 provided on kit 40a is provided in the form of a hook-and-loop fastener (i.e., Velcro strips) that provides for a selective engagement and disengagement of portions thereof from one another to provide for securing of the kit 40a in the storage configuration 48. However, while fastening device 46 is here after referenced as a hook-and-loop fastener 46, it is recognized that another fastening device 46 may instead be utilized in kit 40a, such as a snap-fit fastener or other mechanical fastener. In one embodiment, the hook-and-loop fastener 46 may be provided on outer surface 52 of the housing sheet 42. A first portion of the fastener 46 may be configured as a strip extending outwardly from one end of the housing sheet 42, with a second portion of the fastener positioned on the outer surface 52 of the housing sheet 42 so as to be aligned with and connectable to the first portion upon the housing sheet 42 being rolled up into its storage configuration 48.

In some embodiments, additional information regarding the kit 40a may be applied as a label 68 (or printed on) on the outer surface of housing sheet 42. In one example, an identification label 68 may be adhered to the outer surface of housing sheet 42, with the identification label 68 indicative of the medical supplies included in the kit 40a, as shown in FIG. 4.

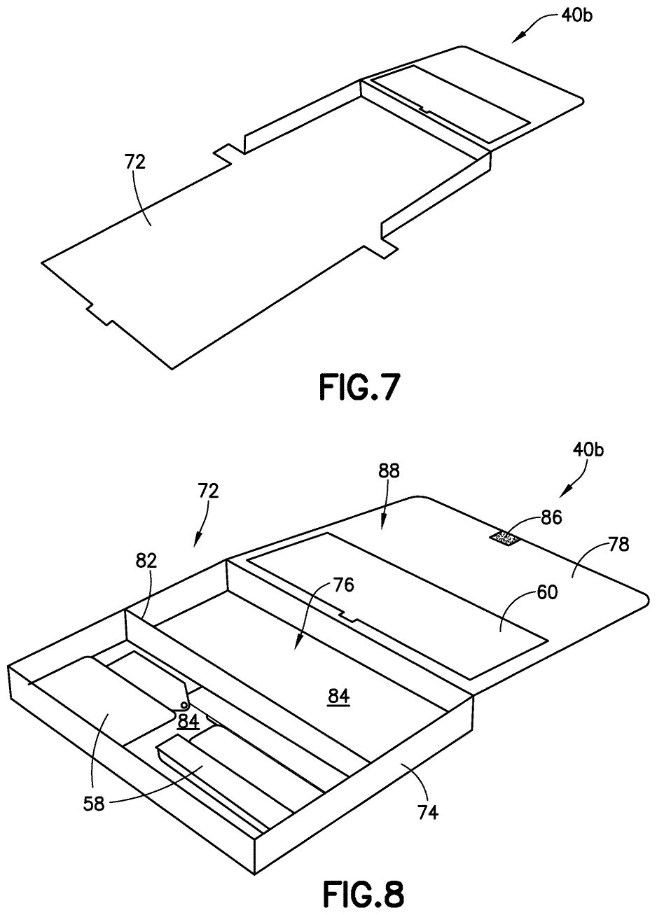

Referring now to FIGS. 7-11, a medical device kit (“kit”) 40b is shown in accordance with another aspect of the disclosure. Again, the kit 40b is described here as catheter kit that can be used in a procedure to replace an old dressing assembly for an indwelling catheter, flush an indwelling catheter, remove an indwelling catheter, or insert a new indwelling catheter. The kit 40b of FIGS. 7-11 is thus described in the present embodiment as a catheter kit and includes various components needed to remove or insert a catheter or perform catheter maintenance (dressing change procedure, catheter flushing procedure, etc.), though it is appreciated that the kit 40b can be configured for one or more of a variety of procedures involving a medical device (e.g., anesthesia kit). The discussion to follow is therefore illustrative and is not to be considered as limiting.

The kit 40b generally includes a housing 72 and a plurality of medical supplies 58 packaged in the housing 72. As can be seen in FIG. 7, the kit 40b is provided initially as an empty housing 72, with the housing in the form of a box-type housing (hereafter “box-type housing 72”). According to embodiments, the box-type housing 72 may be composed of a corrugated fiberboard, paperboard, mat board, or other suitable material that provides structure and rigidity to the housing. In some embodiments, the material of box-type housing 72 (e.g., fiberboard) may be coated with wax or lined with polyethylene, to prevent shedding of fibers of the box material during use of the kit 40b. The box-type housing 72 may be initially provided (i.e., shipped) in a flat configuration and subsequently reconfigured into a three-dimensional shape.

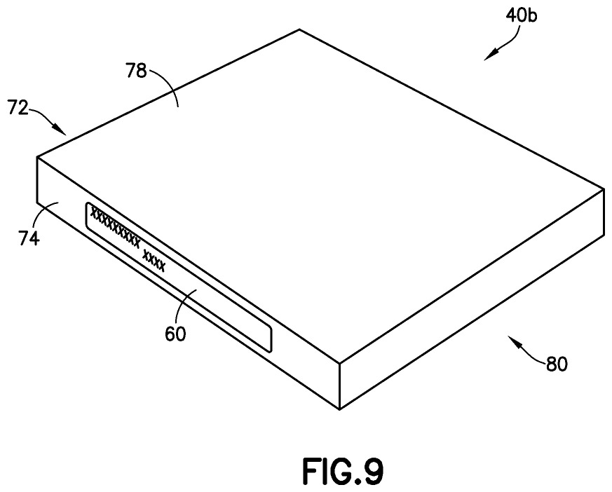

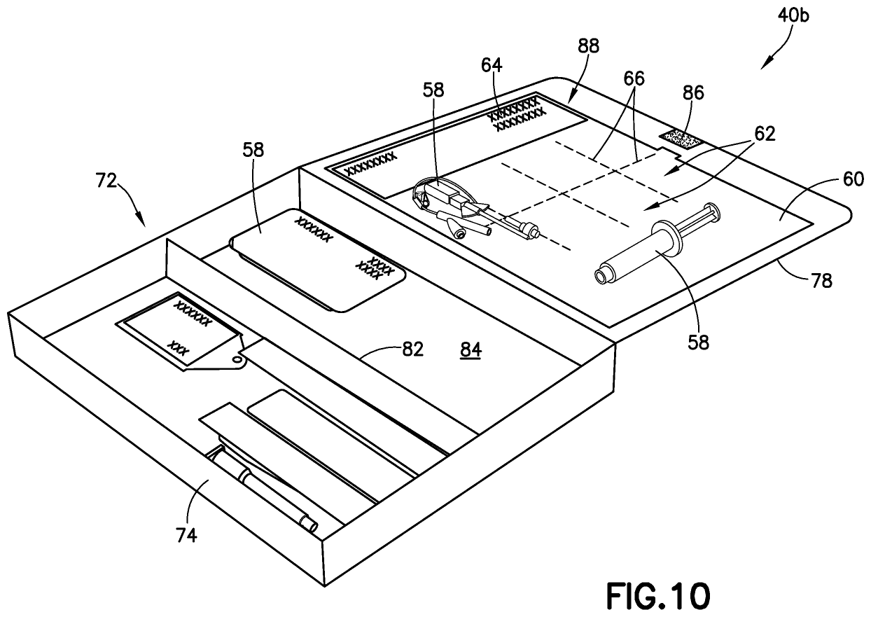

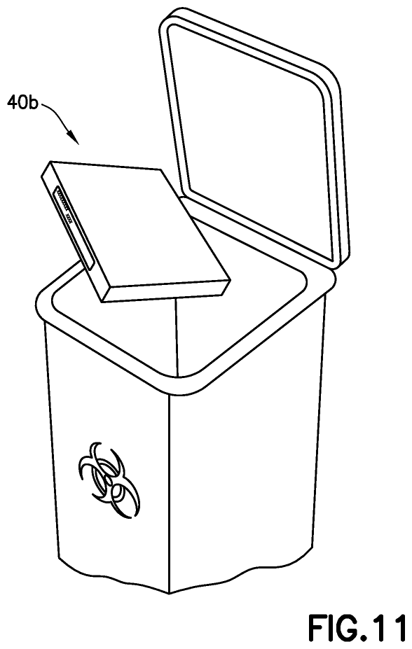

As shown in FIGS. 8 and 10, the box-type housing 72 includes a main housing portion 74 that defines an interior volume 76 of the housing 72, along with and a lid 78 pivotably connected to the main housing portion 74. The lid 78 is movable between an open position and a closed position, with the box-type housing 72 being in a storage configuration 80 when the lid 78 is in the closed position, as shown in FIG. 9. One or more partitions 82 are positioned within the interior volume 76 of main housing portion 74 that define a plurality compartments 84 in the box-type housing 72. The compartments 84 are configured to receive medical supplies 58 therein and provide for the separation and organization of such supplies. In some embodiments, a fastening device 86 may be provided on the box-type housing 72 to secure the lid 78 to the main housing portion 74, with such fastening device 86 being in the form of a snap-type closure or other mechanical fastener or in the form of protrusions on the lid 78 that insert into the main housing portion 74, as examples.

As indicated above, each of the compartments 84 in housing is sized to receive therein medical devices and supplies 58 that are to be used during the procedure to be performed by the clinician. According to embodiments, the devices/supplies 58 may include sterile gloves, alcohol wipes, syringes, gauze, sutures, tape, dressings, disinfecting caps, swab sticks, tourniquets, securement devices, hemostatic agents, and/or anticoagulants, as examples. The components could vary in number, type, position, etc., in other embodiments, however. Similarly, the size, shape, number, and configuration of the compartments 84 themselves can vary from what is shown and described herein. In the present embodiment, the kit 40b includes two (2) compartments 84, though other numbers of compartments 84 can be included.

As shown in FIGS. 7, 8, and 10, a sterile prep sheet 60 may be included in kit 40b that, during usage of the kit 40b, may be removed and unfolded to provide a surface/platform or “prep area” on which the medical devices and supplies 58 included in the kit 40b may be placed and arranged by the clinician upon being removed from the compartments 84. The prep sheet 60 is attached to an inner surface 88 of the lid 78, such as by being adhered or otherwise mechanically fastened to the lid 78, with the prep sheet 60 initially in a folded configuration to prevent an inner surface of the prep sheet 60 from being exposed. In some embodiments, the prep sheet 60 may be subjected to sterilization procedures sufficient to render the prep sheet 60 a sterile field onto which the devices/supplies 58 may be laid (with the devices/supplies 58 also sterilized), while in other embodiments the prep sheet 60 is configured as a clean surface instead of a sterile surface, e.g., the prep sheet 60 is suitable for a procedure where only a clean field is required, as opposed to a sterile field.

FIG. 10 illustrates the sterile prep sheet 60 in an unfolded state and arranged to receive medical supplies 58 thereon from kit 40b. The prep sheet 60 includes thereon a plurality of queuing areas 62 for arranging the medical supplies 58, as well as instructions for use 64 for some or all of the medical supplies 58 in kit 40b. In some embodiments, the queuing areas 62 are provided as a grid-like arrangement of rectangular areas, with the areas demarcated by lines 66 printed directly on the prep sheet 60. The instructions for use 64 of the various medical supplies 58 may be provided above the queuing areas 62. In some embodiments, the instructions for use 64 may be provided in the form of an adhesive label that may secured on the prep sheet 60. The instructions for use 64 may include detailed instructions thereon for the use of some or all of the medical supplies 58 to be arranged in the queuing areas 62. The instructions for use 64 are provided in a sequential arrangement in accordance with an order in which the medical supplies 58 are to be used in the medical procedure—i.e., a series of sequential steps for performing the procedure, with a medical device or component used at each step.

In some embodiments, additional information regarding the kit 40b may be applied as a label 68 (or printed on) on the outer surface of box-type housing 72. In one example, an identification label 68 may be adhered to the outer surface of box-type housing 72, with the identification label 68 indicative of the medical supplies included in the kit 40b, as shown in FIG. 9.

Following now is a work flow describing a plurality of steps to be followed by a clinician or other healthcare worker in providing a medical device kit to be used in performing a desired medical treatment or procedure—such as a kit 40a, 40b shown and described in FIGS. 2-6 and 7-11. In some non-limiting embodiments or aspects, one or more of the steps of the work flow may be performed by a (computing) device including a processor executing software instructions stored by a computer-readable medium, such as a memory and/or other storage component. A computer-readable medium (e.g., a non-transitory computer-readable medium) is defined herein as a non-transitory memory device. A non-transitory memory device includes memory space located inside of a single physical storage device or memory space spread across multiple physical storage devices. Software instructions may be read into the memory and/or other storage component from another computer-readable medium or from another device via a communication interface. When executed, software instructions stored in the memory and/or storage component may cause the processor to perform one or more processes or steps described herein. Additionally or alternatively, hardwired circuitry may be used in place of or in combination with software instructions to perform one or more processes described herein. The device may be capable of receiving information from, storing information in, communicating information to, or searching information stored in the data storage or one or more data structures in the memory and/or storage component 508.

Referring now to FIG. 12, a preparation and usage cycle of a self-packed, customizable medical device kit is shown, in accordance with an embodiment of the disclosure. The cycle or method 90 of FIG. 12 begins at step 92 with the ordering of medical supplies 58 and/or medical device kits 40a, 40b to be used in performing a medical treatment or procedure. The ordering of medical supplies 58 and/or medical device kits 40a, 40b at step 92 may be an automated or semi-automated process performed, in whole or in part, by a computing device and/or by a clinician or other healthcare worker. For example, according to one embodiment, ordering of medical supplies 58 and/or medical device kits 40a, 40b may be done automatically in response to a detection/determination that a stock thereof is getting low, as determined by an inventory tracking system at the facility.

Regarding the ordering of medical supplies 58, medical devices or components may be individually ordered at step 92, such that only those medical supplies 58 identified as being desired for inclusion in the medical device kit 40a, 40b are ordered. As previously described, exemplary treatments/procedures for which the kit 40a, 40b may be used include changing a catheter dressing assembly flushing an indwelling catheter, removing an indwelling catheter, or inserting a new indwelling catheter, as non-limiting examples, and the medical supplies 58 ordered at step 92 will be determined by the specific medical treatment or procedure for which the kit 40a, 40b will be used.

As indicated above, in addition to the ordering of medical supplies 58, step 92 also includes the ordering of the empty kits 40a, 40b themselves-i.e., the empty housings 42, 72 of the kits 40a, 40b—such that with the empty kits 40a, 40b and medical supplies 58 both ordered, the kits 40a, 40b may be self-packed at the healthcare facility. The ordering of the empty kits 40a, 40b may be an automated or semi-automated process performed in whole or in part by a computing device. In ordering of the empty kits 40a, 40b, a database is first accessed (e.g., stored in memory of the computing device) that includes information on a plurality of kit housing types-with each kit housing type indicates a set of medical supplies that can be stored by a kit housing of the kit housing type.

In ordering of the empty kits 40a, 40b, the computing device also receives an identifier for the treatment facility to which the medical device kits 40a, 40b are to be shipped and determines (based on the identifier), the medical supplies stocked at the treatment facility. That is, medical supplies stored/stocked at the treatment facility (or currently on order for shipping to the facility) may be associated with the identifier. The computing device then operates to identify a kit housing type of the plurality of kit housing types that can store at least a portion of the medical supplies stocked at the treatment facility (i.e., a kit housing type that is configured to store the medical supplies therein). As indicated above, such identifying of a kit housing type that can store at least a portion of the medical supplies stocked at the treatment facility may be performed via accessing of a database (stored in the memory of the computing device or in a database accessed by the computing device) that includes information on a plurality of kit housing types and of the medical supplies that can be stored thereby. Responsive to a proper kit housing type being identified, appropriate empty kits 40a, 40b may be ordered for providing to the treatment facility.

The medical supplies 58 and empty kit housings 42, 72 ordered at step 92 may then subsequently be shipped to the healthcare facility and unpacked thereat (step 94), such that the ordered supplies and kit housings are on hand at the facility and available for packing. In some embodiments, the kit housings 42, 72 may be packed/shipped in a flat configuration (i.e., either the housing sheets 42 or the flat/unassembled box-type housings 72), so as to minimize packing space.

The method continues at step 96, where the empty kit housings 42, 72 are prepped or packed with the ordered medical supplies 58 at the healthcare facility (i.e., the empty kit housings 42, 72 receive medical supplies). The medical supplies 58 are packed in the empty kit housings 42, 72 in a number and arrangement as desired by the healthcare worker. According to one embodiment, where the kit housing is in the form of a housing sheet 42, the medical supplies 58 are inserted into pockets 44 provided on the housing sheet 42. According to another embodiment, where the kit housing is in the form of a box-type housing 72, the medical supplies 58 are inserted into compartments 84 formed in the box-type housing 72.

Also at step 96, a sterile prep sheet 60 is prepared and packed into the kit 40a, 40b. As indicated above, the prep sheet 60 includes thereon a plurality of queuing areas 62 for arranging the medical supplies 58 when the kit 40a, 40b is used. In some embodiments, the queuing areas 62 are provided as a grid-like arrangement of rectangular areas, with the areas demarcated by lines 66 printed directly on the prep sheet 60. In preparing the sterile prep sheet 60, instructions for use 64 may be attached to the prep sheet 60 in an area adjacent the queuing areas 62. The instructions for use 64 may include instructions for the various medical supplies 58 in kit 40a, 40b. In some embodiments, the instructions for use 64 may be provided in the form of an adhesive label (i.e., instructional label) that may secured on the prep sheet 60. The instructions for use 64 may include detailed instructions thereon for the use of some or all of the medical supplies 58 to be arranged in the queuing areas 62. The instructions for use 64 are provided in a sequential arrangement in accordance with an order in which the medical supplies 58 are to be used in the medical procedure—i.e., a series of sequential steps for performing the procedure, with a medical device or component used at each step.

According to embodiments, the prep sheet 60 may be packed or attached to the kit 40a, 40b at step 96. In one embodiment, where the kit housing is in the form of a housing sheet 42, the prep sheet 60 is folded and inserted into a pocket 44 provided on the housing sheet 42. In another embodiment, where the kit housing is in the form of a box-type housing 72, the prep sheet 60 is folded and attached to an inner surface 88 of the lid 78, such as by being adhered or otherwise mechanically fastened thereto.

As a final step of the packing of kit 40a, 40b at step 96, the kit 40a, 40b may be put into its storage configuration 48, 80, such that the kit 40a, 40b may safely house the medical supplies 58 thereon until such time that the kit 40a, 40b is to be used. According to one embodiment, where the kit housing is in the form of a housing sheet 42, the housing sheet 42 may be actuated into a rolled storage configuration 48 and then secured shut via the use of the fastening device 46 provided thereon. According to another embodiment, where the kit housing is in the form of a box-type housing 72, the lid 78 of the box-type housing 72 may be closed and then secured shut via the use of the fastening device 86 provided thereon.

In a next step of method 90, after packing of the kit 40a, 40b is completed, the kit 40a, 40b may be labeled to enable future identification of the contents of the kit 40a, 40b, as indicated at step 98. That is, a label 68 may be attached to the outer surface of the housing of kit 40a, 40b, with the label 68 indicating the medical supplies 58 included in the kit 40a, 40b and/or the procedure for which the kit 40a, 40b is to be used.

At a time when it is desired to use a kit 40a, 40b as part of a medical treatment or procedure, the kit 40a, 40b may be pulled and transported to a desired location at the healthcare facility (step 100) and then subsequently prepped for use at step 102, such as by being prepped at the bedside of a patient. According to embodiments, in unpacking the kit 40a, 40b, the sterile prep sheet 60 may first be unfolded and prepared for use-such as by being laid in a flat configuration in preparation for receiving medical devices or components 58 thereon. Upon the prep sheet 60 being appropriately prepared, the medical supplies 58 are removed from their respective pockets 44/compartments 84 in the kit 40a, 40b, packaging is removed from the individual devices/components 58 (if present), and then the devices/components 58 are laid on the prep sheet 60. As previously described, the medical devices and components 58 may be arranged on the prep sheet 60 in accordance with the instructions for use 64 provided on the prep sheet 60, with the medical devices and components 58 arranged on the queuing areas 62 in accordance with a sequential order in which the devices and components 58 are to be used in the medical procedure.

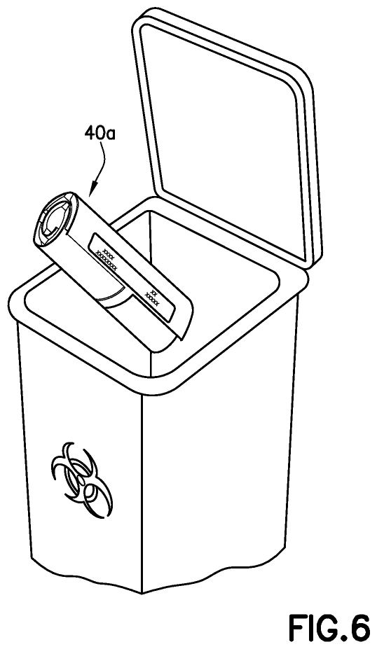

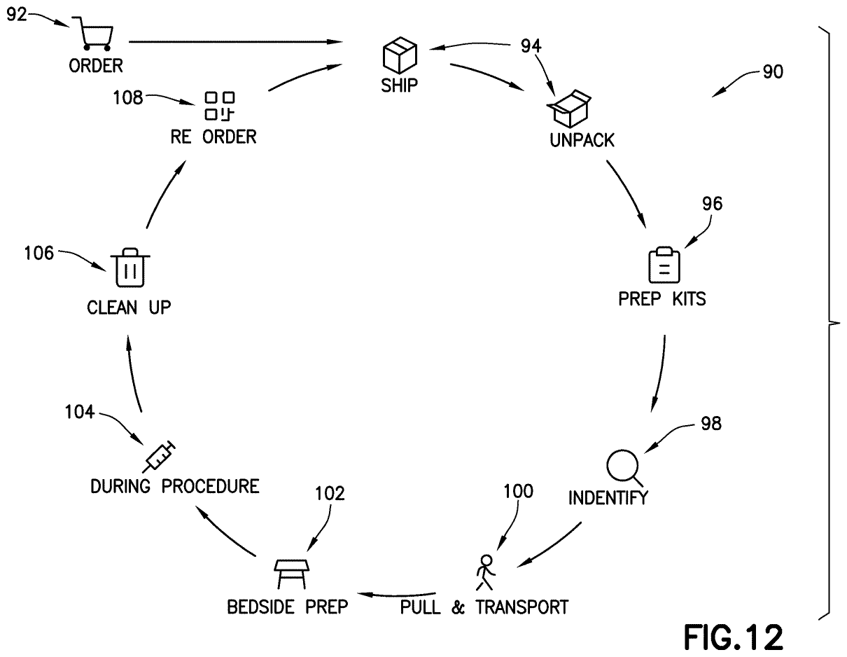

Following bedside prep, the medical procedure may be performed by a clinician at step 104 using the medical supplies 58 included in kit 40a, 40b. Following completion of the procedure, the area around the kit 40a, 40b may be cleaned, as indicated at step 106. That is, used medical supplies 58 from kit 40a, 40b may be gathered and placed back within the housing of the kit 40a, 40b (e.g., within the interior volume 76 of box-type housing 72). The housing of kit 40a, 40b may then be resealed in its storage configuration 48, 80 and subsequently disposed of into a proper waste receptacle, i.e., a hazardous waste receptacle or non-hazardous wasted receptacle, as indicated at FIGS. 6 and 11 for example.

According to embodiments, after usage (and clean-up) of a kit 40a, 40b is complete, an automated inventory tracking system and/or a healthcare worker can determine if additional medical supplies 58 may need to be re-ordered, as indicated at step 108. That is, if any medical device or component 58 that is included in the kit 40a, 40b is running low, the inventory tracking system and/or healthcare worker may place an order for that specific device/component. In some embodiments, a QR code or barcode on a storage bin for a particular medical device or component may be scanned to facilitate re-ordering of that device/component. With re-ordering of individual devices and components provided (rather than reordering pre-packaged kits as a whole) as part of method 90, excess inventory can be reduced at the healthcare facility, thereby saving space at the facility and reducing costs.

Beneficially, embodiments of the medical device kits described herein, and methods for providing such kits, provide for self-packed, customizable kits for use by a clinician. Such self-packed, customizable kits address issues of the waste and disposal of unused devices or components that may occur with the use of pre-packaged medical device kits.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A medical device kit for packaging medical supplies for use in a medical procedure, the medical device kit comprising:
a housing sheet comprising an inner surface and an outer surface, with the housing sheet configurable between a usage configuration and a storage configuration;
a plurality of pockets configured to retain medical supplies therein, the plurality of pockets attached to the inner surface of the housing sheet;
a sterile prep sheet positioned in a pocket of the plurality of pockets, the sterile prep sheet including queuing areas for arranging the medical supplies and instructions for use for some or all of the medical supplies; and
a fastening device configured to secure the housing sheet in the storage configuration.

2. The medical device kit of claim 1, wherein the sterile prep sheet is pre-labeled with the instructions for use, to enable proper arrangement of medical supplies in the medical device kit on the queuing areas.

3. The medical device kit of claim 2, wherein the instructions for use are ordered sequentially, according to an order in which the medical supplies are to be used in the medical procedure.

4. The medical device kit of claim 3, wherein the medical device kit is a customizable, self-packed medical device kit, with the medical supplies comprising a customized collection of medical supplies packed by a user, as determined by the medical procedure.

5. A medical device kit for packaging medical supplies for use in a medical procedure, the medical device kit comprising:
a box-type housing defining an interior volume, the box-type and having a lid movable between an open position and a closed position, with the box-type housing being in the storage configuration when the lid is in the closed position;
one or more partitions positioned within the interior volume that define a plurality compartments in the box-type housing configured to receive medical supplies therein; and
a sterile prep sheet attached to an inner surface of the lid, the sterile prep sheet including queuing areas for arranging the medical supplies and instructions for use for some or all of the medical supplies.

6. The medical device kit of claim 5, wherein the sterile prep sheet is attached to the inner surface of the lid in a folded configuration, so that an inner surface of the sterile prep sheet is not exposed.

7. The medical device kit of claim 5, wherein the sterile prep sheet is pre-labeled with the instructions for use, to enable proper arrangement of medical supplies in the medical device kit on the queuing areas.

8. The medical device kit of claim 7, wherein the instructions for use are ordered sequentially, according to an order in which the medical supplies are to be used in the medical procedure.

9. A method of providing a medical device kit having packaged therein medical supplies for use in a medical procedure, the method comprising:
storing, in a memory, a plurality of kit housing types, wherein a kit housing type indicates a set of medical supplies that can be stored by a kit housing of the kit housing type;
receiving an identifier for a treatment facility;
determining medical supplies stocked at the treatment facility;
identifying a kit housing type of the plurality of kit housing types that can store at least a portion of the medical supplies stocked at the treatment facility; and
providing a kit housing of the kit housing type to the treatment facility.

10. The method of claim 9 further comprising:
providing the kit housing in an empty state, the kit housing comprising a plurality of receptacles provided therein configured to store medical supplies therein;
receiving the medical supplies into respective ones of the plurality of receptacles;
receiving a sterile prep sheet in the kit housing, the sterile prep sheet including queuing areas and instructions for use for some or all of the medical supplies received by the kit housing; and
actuating the kit housing into a storage configuration.

11. The method of claim 10, wherein providing the kit housing comprises providing a housing sheet configurable between a flat position and a rolled position, with the housing sheet being in the storage configuration when in the rolled position, and wherein the plurality of receptacles comprise a plurality of pockets provided on the housing sheet.

12. The method of claim 11, wherein receiving the medical supplies and receiving the sterile prep sheet comprises inserting the medical supplies and the sterile prep sheet into the plurality of pockets.

13. The method of claim 11, wherein actuating the kit housing into the storage configuration comprises securing the housing sheet in the rolled position via securing of a fastening device on the housing sheet.

14. The method of claim 10, wherein providing the kit housing comprises providing a box-type housing having a lid movable between an open position and a closed position, with the box-type housing being in the storage configuration when the lid is in the closed position, and wherein the plurality of receptacles comprise a plurality compartments defined in the box-type housing.

15. The method of claim 14, wherein receiving the medical supplies comprises depositing the medical supplies into the plurality of compartments, and wherein receiving the sterile prep sheet comprises attaching the sterile prep sheet to an inner surface of the lid, the sterile prep sheet attached so as to be in a folded configuration, so that an inner surface of the sterile prep sheet is not exposed.

16. The method of claim 14, wherein providing the kit housing comprises:
providing box-type housing in a flat, unstructured initial state; and
reconfiguring the box into a three-dimensional shape defining the plurality compartments.

17. The method of claim 10, wherein receiving the medical supplies comprises selecting a customized collection of medical supplies to pack in the kit housing, as determined by the medical procedure.

18. The method of claim 10, wherein the queuing areas comprise demarcated areas printed directly on the sterile prep sheet.

19. The method of claim 10, wherein receiving the sterile prep sheet comprises pre-labeling the sterile prep sheet with the instructions for use, with the instructions for use included according to a sequential order in which the medical supplies are to be used in the medical procedure.

20. The method of claim 10, further comprising attaching an identification label to the kit housing, the identification label indicating the medical supplies included in the medical device kit.

21. The method of claim 10, wherein receiving the medical supplies comprises self-packing the kit housing with medical supplies at a healthcare facility at which the medical procedure is to be performed.
